(19) United States
(12) Patent Application Publication
(10) Pub. No.: US 2004/0146530 A1
(43) Pub. Date: Jul. 29, 2004

(54) Avian vaccine effective against infectious bursal disease virus

(76) Inventor: Jagdev M. Sharma, Vadnais, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/471,266

(22) PCT Filed: Jan. 30, 2002
(86) PCT No.: PCT/US02/02589

(51) Int. Cl. A61K 39/295; A61K 39/12

(52) U.S. Cl. 424/202.1; 424/204.1

(57) ABSTRACT

A poultry vaccine is provided having a biological agent or microbial component that is effective in stimulating a protective cellular and humoral immune response to Infectious Bursal Disease Virus (IBDV). Methods are also provided for making the vaccine and for vaccinating poultry by administering such a vaccine.

### BACKGROUND OF THE INVENTION

Commercial chicken flocks in most parts of the world are routinely vaccinated to protect them against environmental exposure to pathogens. Some of the more common viruses that cause disease in domestic poultry include Marek's disease virus (MDV), infectious bursal disease virus (IBDV), Newcastle disease virus (NDV), infectious bronchitis virus (IBV), infectious laryngotracheitis virus (ILTV), avian encephalomyelitis (AEV), chick anemia virus (CAV), fowlpox virus (FPV), avian influenza virus (AIV), reovirus, avian leukosis virus (ALV), reticuloendotheliosis virus (REV), avian adenovirus and hemorrhagic enteritis virus (HEV). These diseases are of economic importance to the poultry industry.

IBDV is a member of the Bimaviridae family whose genome consists of two segments of double-stranded RNA. It causes an acute, highly contagious disease in young chickens. IBDV causes mortality in chickens and induces immunosuppression. Immunosuppressed chickens become more susceptible to Marek's disease, coccidiosis, inclusion body hepatitis, hemorrhagic aplastic anemia, gangrenous dermatitis, infectious chicken anemia agent, salmonellosis and colibacillosis.

The main target cells for IBD viral replication are the actively dividing B lymphocytes. Thus, the virus infection leads to the destruction of lymphoid cells in the bursa of Fabricius and, to a lesser degree, in other lymphoid organs such as cecal tonsils and spleen. The bursa of Fabricius is a unique, primary lymphoid organ in avian species, where B lymphocytes maturate and differentiate. The bursa is the principal reservoir of virus replication, and peak virus titers in the bursa can be detected between three to five days after IBDV infection. In the bursae of chickens infected with IBDV, productive viral replication is often associated with necrosis, apoptosis of lymphoid cells, inflammatory change, atrophy, and hemorrhages.

Chickens infected with IBDV experience suppression in both humoral and cellular immunity. Humoral immunosuppression appears to be associated with IBDV-induced B-cell destruction, while the mechanism of cellular immunosuppression is largely elusive. The acute phase of the disease, often accompanied by variable mortality and immunosuppression, lasts for about a week. The principal cause of economic loss to the industry is virus-induced immunosuppression, both in humoral and cellular immune functions. Flocks experiencing immunosuppression perform poorly and often develop an increased incidence of secondary infections. Certain strains of IBDV are highly pathogenic and may cause excessive mortality.

In the birds that survive the acute phase of the disease, the virus is cleared, the bursal follicles become repopulated with B cells and immune competence is reestablished. The mechanisms that limit virus replication and promote recovery are not known and may involve virus-specific immune responses.

Chicken is the only avian species known to be susceptible to clinical disease and characteristic lesions caused by IBDV. Turkeys, ducks and ostriches are susceptible to infection with IBDV although, thus far, there have been no published reports on the susceptibility of these species to clinical disease. Most commercial chickens get exposed to IBDV early in life. In unprotected flocks, the virus causes mortality and immunosuppression. Although mortality can be quite significant at times, the major economic concern for the poultry industry is the ability of IBDV to cause immunosuppression. Immunosuppressed flocks perform poorly and show reduced economic return. Both broiler and layer flocks are vulnerable to the immunosuppressive effects of the virus and must be protected by vaccination. Protection against IBDV is believed to be mediated primarily by anti-IBDV antibodies. IBDV vaccines used in commercial flocks are selected by the ability of the vaccines to induce vigorous antibody responses.

Despite widely used vaccination programs, IBD is one of the major economically important diseases of poultry industry world-wide. The disease causes recurring economic loss. Currently, it is estimated that 20-40 billion doses of IBDV vaccine are sold yearly worldwide. Despite extensive vaccination, IBD outbreaks continue to occur.

Thus, there is an unmet need for an improved, effective vaccine against IBDV that protects the birds against the disease and associated immunosuppression. A vaccine that would generate a humoral and a strong cellular immune response meets this need.

### SUMMARY OF THE INVENTION

The present invention provides a poultry vaccine that is an attenuated infectious bursal disease virus (IBDV), wherein the vaccine is effective in inducing immunological protection against IBDV infection and is non-pathogenic. As used herein the term “attenuated” is defined as being rendered less virulent in a non-chemically-induced or genetically engineered manner. In particular, in this specification “attenuated” means passaged in tissue culture. The term “non-pathogenic” is used herein to mean non-virulent or unable to induce illness. The term “cell-adapted” is defined herein to mean virus that is transferred or passaged from one culture of cells to an next culture of cells.

The present invention provides a poultry vaccine that contains a biological agent or microbial component that is effective in inducing improved protection against IBDV by stimulating a strong cellular response in addition to a strong humoral immune response as compared to traditional IBDV vaccines. The live biological agent of the vaccine may be a virus. For example the virus may be an attenuated virus, a recombinant virus or a virus that has been altered by chemical, physical or molecular means. The terms “traditional” or “conventional” are used to refer to currently available IBDV vaccines. The new IBDV vaccine of the present invention is different from conventional vaccines because the new vaccine is selected on its ability to generate anti-IBDV cellular immunity. The conventional vaccines are selected on their ability to generate humoral immunity without an assessment of their ability to generate cellular immunity.

IBDV vaccines are generally classified by those skilled in the art as being “mild,” “intermediate” or “hot.” These terms refer to the residual virulence of the vaccine virus. Thus, a “mild” vaccine would retain a minimal, if any, virulence, and a “hot” vaccine would be highly virulent. In general, however, a mild vaccine would not generate a protective immune response. Conventional intermediate and hot vaccines are known to cause variable, often extensive, bursal necrosis and associated immunosuppression. The conventional vaccines induce poor protection in chicks bearing anti-IBDV antibodies such as newly hatched progeny of IBDV-exposed hens. The new vaccine causes reduced bursal damage and fewer immunosuppressive side effects, and is more effective in the presence of pre-existing antibody (such as maternal immunity) in providing protective immunity to the animal than conventional vaccines.

The vaccines of the present invention is effective in such avian species as chicken, turkey, duck or ostrich, in particular chicken. In one embodiment, the vaccine is IBDV that has been attenuated in macrophage cells. The vaccine may further contain more than one strain of IBDV, or may contain additional biological agents or microbial components effective against a second disease to form a multivalent vaccine. For example, the vaccine may contain a mixture of “mild” or “intermediate” virus in addition to “hot” virus. This mixture may be in the ratio of, including but not limited to, 1:99, 25:75, 50:50, 80:20, 90:10, 95:5, 99:1 or 99.9:0.1 intermediate or mild to hot virus. The vaccine may contain one or more adjuvants.

The present invention also provides a method of protecting poultry by administering to the poultry an immunologically protective amount of a vaccine of the present invention. As used herein, the term “immunologically protective” means that the vaccine is effective in inducing an immune response. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

The present invention also provides a method of making an attenuated IBDV vaccine. The vaccine is prepared by passaging IBDV in macrophage cells for at least 2, 5, 10 or 13 passages. In particular, the IBDV may be passaged for about 30, 20, 16 or 15 passages. For example the IBDV may be passaged for 10 to 16 passages, or 13 to 15 passages. The cells used in the tissue culture passaging may be poultry macrophage cells, such as those from chickens. For example, the cells may be the MQ-NCSU cell line.

### DETAILED DESCRIPTION

Management of IBDV in commercial flocks is difficult, with only certain “vaccination windows” available when chicks can be effectively provided with immunity, first via maternal antibodies accessed through the egg yolk, then through drinking water or spray between 0 and 4 weeks of life, again through drinking water or spray between 8 and 10 weeks of age, and with injected vaccines at 16 to 18 weeks, followed by periodic adult immune system checks and re-injection if necessary. During the first 0 to 4 weeks, the chicks do not accept the vaccine effectively due to transmitted maternal immunity. A vaccine with improved cell-mediated immunity that could be administered at any age could help protect chicks during some of the “windows” where they are currently vulnerable and traditional vaccines are less effective.

An improved IBDV vaccine that stimulates the chick's own cellular immune system (T cells) in addition to humoral immune system would be more effective, unique, and highly desirable, leading to a more efficacious vaccination process.

All previously available IBDV vaccines were selected on their ability to generate a strong humoral response. The present research has shown that cellular immunity is also critical for protection against IBDV. A vaccine that generates a strong cellular immunity as well as humoral immunity is needed. The protective efficacy of such a vaccine, especially if the vaccine is effective in the face of maternal antibody, would be much better than the presently existing vaccines.

Molecular technology and immunohistochemistry have been used by the present inventors to learn that bursectomized chickens allow the replication of IBDV in a number of non-bursal tissues. Several serial passages of the virus have been made in bursectomized chickens. This virus is valuable as a vaccine because it has reduced ability to destroy the bursa and thus has reduced immunosuppressive side effects.

We have found that chickens exposed to IBDV mount a vigorous T-cell response. Following IBDV infection, there is a dramatic influx of highly activated, virus-specific T-cells in the bursa. Bursa normally contain very few T-cells. We propose that T-cells are involved in viral clearance, recovery and protective immunity to IBDV. We have developed a T-cell immunodeficiency model to confirm that T-cells play a role in the pathogenesis and control of IBDV.

Several plasmid vectors have been constructed with IBDV and cytokine gene inserts. The cytokine genes include types I and II interferons. The efficacy of these plasmid in inducing protective immunity in chickens has been investigated. Cytokine containing recombinant IBDV initiated a better immune response than IBDV lacking such gene inserts. These data provide information required to construct the appropriate recombinant vaccines.

The present invention provides a poultry vaccine that contains a biological agent or microbial component that is effective in stimulating an improved protective cellular and humoral immune response to IBDV as compared to traditional IBDV vaccines. The vaccine is effective in such avian species as chicken, turkey, duck or ostrich, in particular chicken. The live biological agent of the vaccine may be a virus. For example the virus may be an attenuated virus, a recombinant virus or a virus that has been altered by chemical, physical or molecular means. The vaccine may further contain more than one strain of IBDV or additional biological agents or microbial components effective against other diseases to form a multivalent vaccine. The vaccine may contain one or more adjuvants.

The present invention also provides a method of protecting poultry by administering a vaccine that is effective in inducing cellular and humoral immunity and that contains a biological agent or microbial component that is effective in stimulating a protective cellular and humoral immune response to IBDV.

The vaccine of the present invention can be administered via conventional modes of administration or in ovo. Methods of in ovo immunization are set forth, for example, in U.S. Pat. No. 6,048,535. Vaccination can be performed at any age. For in ovo vaccination, vaccination would be done in the last quarter of embryonal development but may be done at any time during embryonation. The vaccines according to the invention can, for example, be administered intramuscularly, subcutaneously, orally, intraocularly, intratracheally, intranasally,in ovo, in drinking water, in the form of sprays or by contact spread. Preferably, chickens are given the first vaccine in ovo or at one day of age. Subsequent vaccinations are done according to need. Breeder chickens can be vaccinated before and during the lay cycle (several inoculations).

Vaccines are often formulated and inoculated with various adjuvants. The adjuvants aid in attaining a more durable and higher level of immunity using small amounts of antigen or fewer doses than if the immunogen were administered alone. The mechanism of adjuvant action is complex, and may involve the stimulation of cytokine production, phagocytosis and other activities of the reticuloendothelial system as well as a delayed release and degradation of the antigen. Suitable adjuvants include but are not limited to surfactants, e.g., hexadecyl amine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine formulation, or may be conjugated to proteins such as keyhole limpet hemocyanin (KLH) or human serum albumin (HSA) or other polymers.

Vaccines containing live virus can be prepared and marketed in the form of a suspension, or lyophilized. Lyophilized vaccines can preferably contain one or more stabilizers. Suitable stabilizers are, for example, SPGA (Bovarnik (1950): J. Bacteriology 59; 509), carbohydrates (such as sorbitol, mannitol, starch, sucrose, dextran or glucose), proteins (such as albumin or casein), or degradation products thereof, protein-containing materials (such as bovine serum or skimmed milk) and buffers (such as alkali metal phosphates).

The aim of inactivation of IBD viruses is to eliminate both reproductive ability and virulence of the viruses. In general, this can be achieved by chemical or physical means. Chemical inactivation can be effected by treating the viruses with, for example, enzymes, formaldehyde, β-propiolactone, ethylene-imine or a derivative thereof, an organic solvent (such as a halogenated hydrocarbon) and/or a detergent (such as TWEEN, TRITON X, sodium desoxy-cholate, sulphobetain or cetyl trimethylammonium salts). If necessary, inactivating substance is neutralized afterwards; material inactivated with formaldehyde can, for example, be neutralized with thiosulphate. Physical inactivation can preferably be carried out by subjecting the viruses to energy-rich radiation, such as UV light, X-radiation or γ-radiation. If desired, the pH can be brought back to a value of about 7 after treatment. ***

Usually, an adjuvant, and, if desired, one or more emulsifiers, such as TWEEN and SPAN, may also be added to the inactivated virus material.

The invention also includes combination vaccines, where the IBDV material according to the present invention is combined with one or more additional homologous or heterologous biological agents effective against various diseases.

The vaccines according to the invention are suitable for protecting poultry (such as chickens and turkeys) against IBD (Gumboro's disease). The following examples are intended to illustrate but not limit the invention.

### EXAMPLES

### Example 1

### Development of an IBDV Vaccine that Causes Reduced Damage to the Bursa Yet Replicates in the Bird and Induces Strong Immunity

Data indicate that serial passaging of IBDV in bursectomized chickens or non-B cells in vitro has selected a virus that replicates preferentially in non-bursal sites in the birds. These viruses were propagated in chicken embryos, chicken embryo fibroblast (CEF), or macrophage cell lines. The viruses were then cloned. The cloned or uncloned viruses were tested for (1) induction of cellular or humoral immunity; (2) protective efficacy following in ovo or post-hatch administration; (3) onset and duration of protection, (4) potential for causing pathological lesions and immunosuppression; (5) protective ability in antibody-positive birds, and (6) physical and molecular characteristics.

A virulent or intermediate isolate of IBDV is chemically mutated using established procedures. Viral mutant are screened and the mutant(s) that have lost virulence but have retained immunogenicity are identified. A virulent or intermediate isolate of IBDV is genetically cloned and site-directed mutations are induced in the genome. The mutant virus has enhanced immunogenic potential.

### Example 2

### Reduction of Pathogenicity and Enhancement of Immunogenicity of Intermediate IBDV Vaccine

The effects of concurrent in ovo or post-hatch administration of cytokine-inducing chemical or compounds and intermediate IBDV are examined. A commercially available inhibitor of iNOS gene is also examined. This gene codes for nitric oxide. Recent data have show that nitric oxide is responsible for much of the pathological lesions induced by intermediate IBDV and the use of the inhibitor prevents lesions.

### Example 3

### Preparation of a Recombinant Vaccine Vectored in a Bacterial Vector

Viral genes in concert with cytokine genes are incorporated in bacterial plasmids. The recombinant plasmids are used for immunizing chickens. The viral gene plus cytokine gene combinations that produce the most desirable immune response are used as vaccines. These selected combinations are used to construct live recombinant vaccines in viral vectors.

### Example 4

### Combination Vaccines

A “mixed” or combination vaccine is an alternative approach to protecting maternal antibody-bearing chicks against IBD. The efficacy of a mixture containing a minimum infectious dose of a high virulence virus and a high dose of a low virulence virus as an effective vaccine in maternal antibody positive birds was examined. The idea was that in chicks that have high antibody levels, the high virulence virus was likely to break through the antibody and initiate immunity. In chicks with low or negligible antibody levels, although the high virulence virus would home to the bursa and initiate infection, the presence of an overwhelming dose of the low virulence virus would reduce the pathogenic effects of the high virulence virus.

The preliminary data showed that less that 10 embryo lethal dose₅₀ (ELD₅₀) of IM-IBDV (high virulence virus) can induce bursal lesions in SPF chickens. This dose is being tried in association with 10⁶ tissue culture infectious dose₅₀ (TCID₅₀) of a mild strain of IBDV.

IBDV isolates of varying pathogenicity are mixed to create combinations that have the lowest immunosuppressive activity but are highly immunogenic and induce strong cellular or humoral immune responses. For example, the vaccine may contain a mixture of “mild” or “intermediate” virus in addition to “hot” virus. This mixture is in the ratio of, but not limited to, 1:99, 25:75, 50:50, 80:20, 90:10, 95:5, 99:1, or 99.9:0.1 intermediate or mild to hot virus.

### Example 5

### Attenuated Vaccines

Other research groups have attempted to attenuate/adapt IBDV to macrophage cell lines, but were unsuccessful. (See, Abdel-Alim, G and Y. M. Saif. 2001. Imunogenicity and antigenicity of very virulent strains of infectious bursal disease virus. Avian Diseases 45:92-101.)

The present inventor, however, was able to successfully adapt virulent, intermeidate and mild IBDV to replicate in a chicken macrophage cell line. The virulent strain, Irwin Moulthrop (IM) of IBDV was attenuated in chicken macrophage cell line (MQ-NCSU). The adapted virus, designated IM-IBDV-Mac, caused lytic infection in cells and replicated to high titers. IM-IBDV-Mac also replicated in chicken embryo fibroblasts and vero cells, where it induced typical cytopathic effects. The adaptation of IM-IBDV to macrophages resulted in the loss of the ability of the virus to cause mortality in embryonated eggs or chickens but retained its immunogenic potential.

The inventor identified the potential value of microphage-adapted IM-IBDV as a vaccine. The data indicate that the replication of microphage-adapted virus in chicken embryo fibroblast (CEF) cells began at the fourteenth passage (P14). P14 or later passages, but not earlier passages, replicated vigorously in CEF. The replication in CEF became more vigorous with increasing passages in microphage cells. The inventor also discovered that although the virus at P14 in microphage began to replicate and cause cytopathic effect (CPE) in CEF, the first evidence of CPE in microphage cells did not become apparent until P16. It should be noted that PCR was performed on the nucleic acid in the microphage cells at each passage to determine if IBDV was present in the cells, even if the virus did not cause CPE. The results of the PCR analysis indicated that IBDV was indeed present in the cells at all the tested passages. The in vivo characterization of the virus at various passage levels is presented below:

TABLE 1
Summary of characteristics of IM-IBDV adapted
to replicate in MQ-NCSU cells

| Passage | Egg | Chick | Gross | Micro | Antigen | | | Anti- | Pro- |
|---|---|---|---|---|---|---|---|---|---|
| # | mort | mort | bursa | bursa | B | S | Th | body | tection |
| 0 | + | + | + | + | + | + | ND | ++++ | + |
| 10 | + | − | + | + | | | ND | ++++ | ND |
| 13 | ND | − | + | + | + | +/− | ND | ++++ | + |
| 14 | ″ | − | + | + | + | +/− | ND | ++++ | + |
| 15 | ″ | − | + | + | + | +/− | ND | +++ | + |
| 16 | − | − | − | +/− | +/− | +/− | ND | − | − |
| 18 | ND | − | − | +/− | +/− | +/− | ND | − | − |
| 20 | ″ | − | − | +/− | +/− | +/− | ND | − | − |

ND = Not done

Explanation of Results Shown in the Table 1:

Passage #: The number of times IM-IBDV was serially passed in macrophage cells (MQ-NCSU cells). The serial passages were made by freeze/thawing cells and medium from the previous passage, centrifuging the mixture to clear debris, and inoculating a new batch of growing microphage cells. The passages were made every 3 to 7 days. The first evidence of the cytopathic effect in microphage cells was observed at around the 16^{th} passage.

Egg mort.: The ability of the virus to cause embryo mortality when inoculated in 10- to 12-day-old embryonated chicken eggs. Generally, virulent and egg-adapted viruses tend to cause high egg mortality.

Chick mort.: The ability of the virus to cause mortality when inoculated in hatched chickens. SPF chickens were tested at 2-4 weeks of age.

Gross bursa: Chickens exposed to the virus had grossly detectable bursal lesion typical of IBDV. Virulent viruses cause extensive gross bursal lesions.

Micro. Bursa: The ability of the virus to cause microscopic bursal lesions characteristic of IBDV. The ability of a virus to cause microscopic lesions is generally associated with the extent of viral replication in the bursa (B cells). Virulent viruses replicate extensively in the bursa; attenuated viruses replicate less extensively, depending upon the degree of attenuation.

Antigen B S Th: Sections of bursa (B), spleen (S) and thymus (Th) were examined by immunohistochemistry for the presence of virus antigen. ±means that the presence of viral antigen was equivocal. Presence of viral antigen in the spleen and thymus may indicate that the virus is capable of extra-bursal replication and may induce immunity without causing excessive bursal destruction.

Antibody: Chickens were examined for antibody levels two weeks after inoculation with the virus. Antibody levels are shown as very high (++++) to undetectable (−). Highly immunogenic (protective) viruses induce high levels of antibody.

Protection: Birds were exposed to the virus and two weeks later, challenged with virulent virus (P0 of IM-IBDV). “+” indicates protection against mortality and lesion induction by the virulent virus.

General Conclusions from the Table 1:

The results in Table I above indicate that passages 13-15 were good vaccines. These passages have lost the ability to cause mortality in eggs and chickens, but have retained the ability to induce antibodies and protection. Passage levels 16 or greater appear to be too attenuated to serve as effective vaccines.

All publications are incorporated by reference herein, as though individually incorporated by reference. The invention has been described with reference to various specific and preferred embodiments and techniques. However, it should be understood that many variations and modifications may be made while remaining within the scope of the invention.

References:

Abdel-Alim, et al. Avian Diseases 45:92-101

Kim I J et al., 78th Conference of Research Workers in Animal Diseases Nov. 10-11, 1997

Kim I J et at, J. Vir. 74:8884-8892 (October 2000)

Kim I J and J M Sharma, Vet. Immunology and Immunopathology, 74:47-58 (2000)

Rautenschlien S, et al., Abstract for AVMA/AAAP, Salt Lake City, 2000

Sharma J M et al., Devel. Compar. Immunol., 24:223-235 (2000)

Sharma J M et al., Abstract for ANECA, Cancun, Mexico, May 3-5, 2000

Sharma J M et al., Abstract for AVMA/AAAP, Salt Lake City, 2000

Tanimura N and J M Sharma, Avian Diseases 41:638-645 (1997)

Yeh H Y and J M Sharma, Abstract for 136th AVMA Annual Convention (1999)

Yeh H Y et al., Poster for AVMA/AAAP (2000)

What is claimed is:

1. An avian vaccine comprising a cell-adapted infectious bursal disease virus (IBDV), wherein the vaccine is effective in inducing immunological protection against IBDV infection and is non-pathogenic.

2. The vaccine of claim 1, wherein the avian is chicken, turkey, duck or ostrich.

3. The vaccine of claim 2, wherein the avian is chicken.

4. The vaccine of claim 1, wherein the vaccine comprises an additional virus to generate a mixture of viruses.

5. The vaccine of claim 4, wherein the additional virus is an attenuated or unattenuated virus.

6. The vaccine of claim 4, wherein the additional virus is a hot virus.

7. The vaccine of claim 6, wherein the attenuated virus and the hot virus are present in the vaccine in a ratio of 1:99.

8. The vaccine of claim 6, wherein the attenuated virus and the hot virus are present in the vaccine in a ratio of 25:75.

9. The vaccine of claim 6, wherein the attenuated virus and the hot virus are present in the vaccine in a ratio of 50:50.

10. The vaccine of claim 6, wherein the attenuated virus and the hot virus are present in the vaccine in a ratio of 80:20.

11. The vaccine of claim 6, wherein the attenuated virus and the hot virus are present in the vaccine in a ratio of 90:10.

12. The vaccine of claim 6, wherein the attenuated virus and the hot virus are present in the vaccine in a ratio of 95:5.

13. The vaccine of claim 6, wherein the attenuated virus and the hot virus are present in the vaccine in a ratio of or 99:1.

14. The vaccine of claim 6, wherein the attenuated virus and the hot virus are present in the vaccine in a ratio of 99.9:0.1.

15. The vaccine of claim 4, wherein the additional virus is a recombinant virus.

16. The vaccine of claim 4, wherein the additional virus has been altered by chemical, physical or molecular means.

17. The vaccine of claim 4, wherein the additional virus has similar or varying antigenic or pathogenic potential.

18. The vaccine of claim 1, further comprising a biological agent or microbial component effective against a second disease to form a multivalent vaccine.

19. The vaccine of claim 1, further comprising an adjuvant.

20. A method of protecting poultry comprising administering to a bird an immunologically protective amount of the vaccine of claim 1.

21. A poultry vaccine comprising a biological agent or microbial component that is effective in inducing protection against infectious bursal disease virus (IBDV) by stimulating a stronger cellular and humoral immune response to IBDV as compared to a traditional IBDV vaccine.

22. The vaccine of claim 21, wherein said avian species is chicken, turkey, duck or ostrich.

23. The vaccine of claim 22, wherein said avian species is chicken.

24. The vaccine of claim 21, wherein the live biological agent is a virus, or a mixture of viruses.

25. The vaccine of claim 24, wherein the virus is an attenuated virus, or a mixture of attenuated viruses, or a mixture of attenuated and unattenuated viruses.

26. The vaccine of claim 24, wherein the virus is a recombinant virus.

27. The vaccine of claim 24, wherein the virus has been altered by chemical, physical or molecular means.

28. The vaccine of claim 21, further comprising an additional biological agent or microbial component effective against a second disease to form a multivalent vaccine.

29. The vaccine of claim 21, further comprising an adjuvant.

30. A method of protecting poultry comprising administering to a bird an effective cellular and humoral immunity producing amount of the vaccine of claim 21.

31. A method of making an attenuated IBDV vaccine comprising passaging IBDV in macrophage cells for at least 2 passages.

32. The method of claim 31, wherein the IBDV is passaged for at least 5 passages.

33. The method of claim 31, wherein the IBDV is passaged for at least 8 passages.

34. The method of claim 31, wherein the IBDV is passaged for at least 10 passages.

35. The method of claim 31, wherein the IBDV is passaged for less than 30 passages.

36. The method of claim 31, wherein the IBDV is passaged for less than 20 passages.

37. The method of claim 31, wherein the IBDV is passaged for 10 to 16 passages.

38. The method of claim 31, wherein the IBDV is passaged for 13 to 15 passages.

39. The method of claim 31, wherein the macrophage cells are avian or non-avian cells.

40. The method of claim 31, wherein the macrophage cells are avian cells.

41. The method of claim 31, wherein the macrophage cells are chicken cells.

42. The method of claim 31, wherein the cells are cell line MQ-NCSU.
